(19) United States
(12) Patent Application Publication
(10) Pub. No.: US 2008/0131512 A1
(43) Pub. Date: Jun. 5, 2008

(54) Biodegradable hydrogels

(76) Inventors: Wilhelmus Everhardus Hennink, Waddinxveen (NL); Wendelmoed Nelletha Eleonora Van Dijk-Wolthuis, Leiden (NL)

Correspondence Address: MORRISON & FOERSTER LLP, 12531 HIGH BLUFF DRIVE, SUITE 100, SAN DIEGO, CA, 92130-2040, US

(21) Appl. No.: 11/025,296

(22) Filed: Dec. 28, 2004

Related U.S. Application Data
(63) Continuation-in-part of application No. 10/020,627, filed on Dec. 6, 2001, now Pat. No. 7,060,296.
Application No. 10/020,627 is a continuation of application No. 09/214,306, filed on Jul. 20, 1999, now Pat. No. 6,497,903.
(60) Provisional application No. 60/031,671, filed on Nov. 22, 1996.

(30) Foreign Application Priority Data
Jul. 1, 1996 (EP) 96/2011821.4

(51) Int. Cl. A61K 47/36; C08F 251/00; A61K 47/32

(52) U.S. Cl. 424/487; 424/488; 514/777; 514/772.3; 527/314

(57) ABSTRACT

A prepolymer comprises polymerisable macromolecules, each polymerisable macromolecule comprises a polysaccharide backbone and at least two side units, wherein each of the at least two side units comprises a polymerisable group which is connected to the backbone via at least one group which is hydrolysable under physiological conditions; wherein the polymerisable group of each of the side units is independently selected from optionally alkylated and/or hydroxyalkylated acrylates and acrylamides; and wherein the at least one group which is hydrolysable under physiological conditions is selected from carbonate, lactate, glycolate, and succinate. The invention is also directed to biodegradable hydrogels useful in pharmaceutical compositions, comprising polymerized or co-polymerized prepolymers.

This application is a continuation-in-part of U.S. application Ser. No. 10/020,627, which is a continuation of U.S. application Ser. No. 09/214,306 (now U.S. Pat. No. 6,497,903 B1), which was filed under 35 U.S.C. § 111 claiming priority under 35 U.S.C. § 120 from PCT/NL97/00374 filed Jul. 1, 1997 which designates the U.S. and which claims priority from European Application 96/201821.4 filed Jul. 1, 1996 and from U.S. Provisional Application No. 60/031,671 filed November 1996.

### TECHNICAL FIELD

The present invention relates to prepolymers which are polymerizable to yield biodegradable hydrogels, to methods of making such biodegradable hydrogels from prepolymers, to biodegradable hydrogels, and to pharmaceutical compositions comprising such hydrogels.

### BACKGROUND ART

The fast developments in the field of molecular biology and biotechnology have made it possible to produce a large number of pharmaceutically interesting products in large quantities. For instance, pharmaceutically active peptides and proteins can suitably be used as drugs in the treatment of life-threatening diseases, e.g. cancer, and of several types of viral, bacterial and parasitic diseases; in the treatment of e.g. diabetes; in vaccines, e.g. for prophylactic aims, and for contraception purposes. Especially the specialized biological activities of these types of drugs provide tremendous advantages over other types of pharmaceutics.

To illustrate the fast developments, it has been reported (see e.g. Soeterboek and Verheggen, Pharm. Weekblad 130 (1996) 670-675) that in the United States of America, about 275 bio technological products are in phase IV studies, while more than 500 products are under investigation.

Examples of (recombinant) proteins, which are considered very interesting from a pharmacological point of view, are cytokines, such as interleukins, interferons, tumor necrosis factor (TNF), insulin, proteins for use in vaccines, and growth hormones.

Due to their nature, proteins and proteinaceous products, including peptides, which group of products will be referred to as protein drugs hereinbelow, cannot be administered orally. These products tend to degrade rapidly in the gastrointestinal tract, in particular because of the acidic environment and the presence of proteolytic enzymes therein.

Moreover, to a high extent protein drugs are not able to pass endothelial and epithelial barriers, due to their size and, generally, polar character.

For these reasons, protein drugs have to be brought in the system parenterally, i.e. by injection. The pharmacokinetic profile of these products is, however, such that injection of the product per se requires a frequent administration. For, it is a known fact that proteinaceous material is eliminated from the blood circulation within minutes.

In other words, since protein drugs are chemically and/or physically unstable and generally have a short half-time in the human or animal body, multiple daily injections or continuous infusions are required for the protein drug to have a desired therapeutic effect. It will be evident that this is inconvenient for patients requiring these protein drugs. Furthermore, this type of application often requires hospitalization and has logistic drawbacks.

In addition, it appears that at least for certain classes of pharmaceutical proteins, such as cytokines which are presently used in e.g. cancer treatments, the therapeutic efficacy is strongly dependent on effective delivery, e.g. intra- or peritumoral. In such cases, the protein drugs should be directed to the sites where their activity is needed during a prolonged period of time.

Hence, there is a need for delivery systems which have the capacity for controlled release. In the art, delivery systems consisting of polymeric networks in which the proteins are loaded and from which they are gradually released have been proposed.

More in detail, at present, two major types of polymeric delivery systems can be distinguished: biodegradable polymers and non-biodegradable hydrogels.

Biodegradable polymers, e.g. polylactic acid (PLA) and copolymers of PLA with glycolic acid (PLGA), are frequently used as delivery systems for proteins.

Proteins can be incorporated in pharmaceutical delivery systems, e.g. microspheres, by a variety of processes. In vitro and in vivo, usually a biphasic release profile is observed: an initial burst followed by a more gradual release. The burst is caused by proteinaceous material present at or near the surface of the microspheres and by proteinaceous material present in pores. The gradual release is ascribed to a combination of diffusion of the proteinaceous material through the matrix and degradation of the matrix. Especially for larger proteins diffusion in these matrices is negligible, so that the release depends on the degradation of the polymer. The degradation can be influenced by the (co)polymer composition. A well-known strategy to increase the degradation rate of PLA is co-polymerization with glycolic acid.

Although delivery systems based on biodegradable polymers are interesting, it is very difficult to control the release of the incorporated protein. This hampers the applicability of these systems, especially for proteins with a narrow therapeutic window, such as cytokines and hormones. Furthermore, organic solvents have to be used for the encapsulation of the protein in these polymeric systems. Exposure of proteins to organic solvents generally leads to denaturation, which will affect the biological activity of the protein. Furthermore, the very stringent requirements of registration authorities with respect to possible traces of harmful substances may prohibit the use of such formulations of therapeutic drugs in human patients.

Also hydrogels are frequently used as delivery systems for proteins and peptides. Hydrogels can be obtained by crosslinking a water-soluble polymer yielding a three-dimensional network which can contain large amounts of water. Proteins can be loaded into the gel by adding the protein to the polymer before the crosslinking reaction is carried out or by soaking a preformed hydrogel in a protein solution. So, no (aggressive) organic solvents have to be used to load the hydrogels with protein molecules.

In contrast to the biodegradable polymers, the release of proteins from hydrogels can be easily controlled and manipulated by varying the hydrogel characteristics, such as the water content and the crosslink density of the gel. However, a major disadvantage of the currently used hydrogel delivery systems is that they are not biodegradable. This necessitates surgical removal of the gel from the patient after the release of the protein in order to prevent complications of inclusion of the empty hydrogel material (wound tissue is frequently formed).

Biodegradable hydrogels have been used in the preparation of delivery systems for protein drugs. One of these systems comprises crosslinked dextrans obtained by coupling glycidyl methacrylate (GMA) to dextran, followed by radical polymerization of an aqueous solution of GMA-derivatizad dextran (dex-GMA). In this respect, reference is made to Van Dijk-Wolthuis et al. in Macromolecules 28, (1995), 6317-6322 and to De Smedt et al. in Macromolecules 28, (1995), 5082-5068.

Proteins can be encapsulated in the hydrogels by adding proteins to a solution of GMA-derivatized dextran prior to the crosslinking reaction. It appeared that the release of the proteins out of these hydrogels depends on and can be controlled by the degree of crosslinking and the water content of the gel (Hennink et al., J. Contr. Rel. 39, (1996), 47-57).

Although the described, cross-linked dextran hydrogels were expected to be biodegradable, these hydrogels are rather stable under physiological conditions. This is further elaborated in Example 5. It is shown among other that the dissolution time of dextran hydrogels obtained by polymerization of dextran derivatized with glycidyl methacrylate (DS=4) had a dissolution time of about 100 days. Dextran hydrogels, wherein the dextrans have a higher degree of substitution, did not show any signs of degradation during 70 days, even at extreme conditions.

### DISCLOSURE OF THE INVENTION

The object of the present invention is to provide a slow, sustained or otherwise controlled release delivery system which does not possess one or more of the above-mentioned disadvantages, and especially does not require the use of toxic organic solvents for its preparation, does not show the undesired and uncontrollable burst effects, and do not possess a poorly controllable release behavior. The present invention aims to combine the advantages of both types of known delivery systems, i.e. biodegradability under physiological conditions, with controlled drug release.

The present invention provides safe and easily controllable delivery systems, based on particular biodegradable hydrogels, which increase the potential usefulness of protein drugs for the treatment of various diseases. The risks associated with these drugs, such as bursts in the release profile, and the inconvenience for the patient are reduced, while the therapeutic efficacy of drug treatments using the hydrogels of the present invention is increased.

More in detail, the present invention relates to biodegradable hydrogels comprising bonds which are hydrolysable under physiological conditions. The hydrogels of the present invention comprise a polymeric network having polysaccharide backbones which are interconnected through crosslinking units. Each of said crosslinking units comprises at least one group which is hydrolysable under physiological conditions. Such hydrolysable groups are broken in the human or animal body.

The biodegradable hydrogels can be prepared by polymerizing biodegradable prepolymers. These prepolymers are comprised of polymerisable macromolecules having a polysaccharide backbone and at least two side units per backbone. The side units comprise a polymerisable group which is connected to the polysaccharide backbone via at least one group which is hydrolysable under physiological conditions, which group is a carbonate, lactate, glycolate, or succinate group. The polymerisable group is derived from acrylic acid.

Furthermore, the invention provides a method for the preparation of a hydrogel. According to this method, the prepolymers of the invention are polymerized, optionally in the presence of a bioactive compound, to form a crosslinked, biodegradable hydrogel.

In another aspect, the invention provides pharmaceutical compositions comprising such biodegradable hydrogels.

### BRIEF DESCRIPTION OF THE DRAWINGS

FIG. 1 illustrates the influence of the spacer on the swelling ratio of dextran hydrogels;

FIG. 2 illustrates the influence of the DS on the swelling ratio of dex-lactate-HEMA hydrogels;

FIG. 3 illustrates the influence of the initial water content of dex-lactate HEMA hydrogels on the swelling ratio;

FIG. 4 illustrates the swelling behavior of dex-SA-HEMA hydrogels;

FIG. 5 illustrates the release of IgG from degrading hydrogels (dex-lactate-HEMA, DS 2.5);

FIG. 6 shows a ¹H NMR spectrum of HEMAm-CI;

FIG. 7 shows a ¹H NMR spectrum of dex-HEMAm with a calculated DS of 6.5;

FIG. 8 illustrates the release of BSA from hydrogel cylinders with a DS of approx. 5;

FIG. 9 illustrates the release of BSA from hydrogel cylinders with a DS of approx. 7; and

FIG. 10 illustrates the release of BSA from hydrogel cylinders with a DS of approx. 10.

### DETAILED DESCRIPTION OF INVENTION

According to the present invention, prepolymers are provided which comprise polymerisable macromolecules. Each polymerisable macromolecule has a polysaccharide backbone and at least two side units comprising a polymerisable group which is connected to the backbone via at least one group which is hydrolysable under physiological conditions, which is selected from carbonate, lactate, glycolate, and succinate groups. The polymerisable groups of the side units are independently selected from acrylates and acrylamides, which acrylates and acrylamides may optionally be alkylated and/or hydroxyalkylated.

As used herein, a pre polymer is a polymer or oligomer whose molecules are capable of entering, through reactive groups, into further polymerisation. The prepolymers are predominantly composed of polymeric macromolecules. These macromolecules have a polysaccharide backbone, or main chain. A polysaccharide is a polymer whose molecules are composed of multiple units of monosaccharides. Examples of polysaccharides are native and derivatized starch, cellulose, amylose, dextran, dextrin, pectin, carrageen, agar-agar, and alginic acid. Preferred polysaccharide backbones according to the invention are water-soluble species, such as water-soluble starches, cellulose derivatives, e.g. hydroxypropyl cellulose, or dextran. The polysaccharide may be branched or unbranched.

In one of the preferred embodiments, the polysaccharide is a dextran or dextran derivative. Dextran is a polysaccharide produced by chemical synthesis or by bacteria (e.g., by the genera Lactobacillus, Leuconostoc and Streptococcus) from sucrose. The backbone consists of α-1,6 linked D-glucopyranose units with branching at mainly α-1,3 position and a low percentage of α-1,2 and α-1,4 side chains. Dextrans with low molecular weight, dextran T40 and T70 (Mw 40 kDa and 70 kDa, respectively), have been clinically used for more than five decades as blood plasma expanders, to improve peripheral blood flow and as thrombolytic agents. Thus, dextran is safe and highly acceptable for parenteral administration, even in relatively high doses.

The polymerisable macromolecules have at least two side units which are both polymerisable and hydrolysable under physiological conditions. More specifically, they comprise a polymerisable group which is attached to the backbone via at least one hydrolysable group. In the context of the present invention, the terms “hydrolysable” and “hydrolysable under physiological conditions” are used interchangeably unless indicated otherwise, and relate to the capability of a molecule of becoming degraded by non-enzymatic hydrolysis in a native or simulated physiological fluid at body temperature. Typically, substantial hydrolysis occurs within hours, days, weeks, months, or a few years at the most.

The hydrolyzable groups are selected from carbonate, lactate, glycolate, and succinate units. As used herein, a carbonate group or unit is a divalent CO a unit. A lactate or glycolate group or unit is the divalent unit derived from lactic or glycolic acid as it is present in poly(lactic acid) or poly(glycolic acid), respectively. Similarly, a succinate group or unit is a divalent unit derived from succinic acid. All these groups have been used in monomeric, oligomeric, or polymeric form in humans for a significant time, and therefore they can be considered safe.

In a preferred embodiment, the hydrolysable groups are represented by one single carbonate group per side unit, through which the polymerisable group is attached to the polymer backbone. In another preferred embodiment, the side units comprise more than one hydrolysable group, such as a carbonate and one or two lactate units.

The polymerisable groups are ethylenically unsaturated and capable of undergoing radical polymerization. According to the invention, they are selected from—optionally alkylated and/or hydroxy alkylated—acrylates and acrylamides. Examples of suitable polymerisable groups are methacrylate, hydroxyethylmethacrylate, hydroxypropyl methacrylate, hydroxyethyl methacrylamide, and hydroxypropyl methacrylamide groups. As used herein, these names denote the molecular species from which the respective polymerizable groups are derived. Obviously, a hydroxyalkyl acrylate or—acrylamide substituent may no longer comprise its original hydroxyl group if that has been reacted with a hydrolysable group to become attached to the polymer backbone.

Of course, other side units which comprise no polymerisable group and/or no hydrolysable group may also be present in the polymerisable macromolecules.

The prepolymers can be characterized by known techniques, e.g. NMR and IR spectroscopy, differential scanning calorimetry (DSC), and gel permeation chromatography (GPC). In order to couple a polymerisable group such as hydroxyethylmethacrylate (HEMA)—optionally linked to a monomeric or oligomeric lactate and/or glycolate unit—to a polysaccharide such as dextran, the terminal hydroxyl group of the substituent has to be activated. Preferably, the binding to the polysaccharide is affected by carbonyldiimidazole (CDI) as coupling agent. However, also other activation methods can be used. For example, reaction of the hydroxyl function of the substituent with succinic anhydride, fallowed by activation of the formed carboxylic group using established methods (e.g. dicyclohexylcarbodiimide (DCC) activation). The latter method yields prepolymers in which only hydrolytically instable ester bonds age present, which provide different degradation characteristics compared to the dextran derivatives synthesized with the CDI-method, in which both ester bonds and carbonate bonds are present.

The activated substituent, such as HEMA-oligo-lactate-carbonyl-imidazole, is subsequently coupled to the polysaccharide in a suitable aprotic solvent (such as DMSO). This can be done, optionally, in the presence of a catalyst, e.g. a base such as N,N-dimethylaminopyridine (DMAP) or triethylamine. However, it has been found by the inventors that it is also possible to conduct the coupling reaction without any catalyst at all, which has the advantage that no residual catalyst has to be specified or removed from the product before the prepolymer can be used as a starting material for medicinal products. The degree of substitution (i.e. number of moles of methacrylate groups containing prepolymer per 100 moles glucose units of dextran) can be tailored by the ratio of HEMA-containing prepolymer versus polysaccharide in the reaction mixture.

The prepolymers of the invention can be polymerized by radical polymerization to make hydrogels. A hydrogel is a hydrophilic, three-dimensional, crosslinked polymeric network capable of swelling in water. In their swollen state, hydrogels may typically contain from about 20 to more than 99 wt. % of water.

Hydrogels are an important class of materials for tissue engineering and for the controlled release of pharmaceutically active compounds such as therapeutic proteins. The polymeric networks are obtainable by chemically or physically crosslinking hydrophilic polymers. In chemically crosslinked hydrogels, the polymers are connected primarily by covalent bonds. In physically crosslinked gels, the network is formed by physical interactions between different polymer chains, such as by electro static interactions. Accordingly, the respective hydrogels are sometimes termed physical hydrogels, whereas chemically crosslinked hydrogels are also called chemical hydrogels. The hydrogels of the peasant invention are chemical hydrogels, as their crosslinks are covalent, and are made from polymerizing the polymerizable groups of the side units of the prepolymers.

The polymerisation may be carried out using a suitable initiator system, such as a tertiary amime and a persulphate (see e.g. Van Dijk-Wolthuis et al. in Macromolecules 28, and Hennink et al. in J. Contr. Rel. 89; both references being incorporated herein by reference). Depending on the type of prepolymer and on the specific initiator system which is used, the reaction can be designed as a photopolymerization process. It is also possible to use polymerization by gamma irradiation, with the advantage that no initiator and/or catalyst residues have to be extracted from the hydrogel.

The hydrogels obtained by polymerizing one or more prepolymers as defined above are characterized by a structure of polysaccharide backbones which are interconnected through crosslinking units. These crosslinking units, which are derived from the side units of the prepolymer(s), comprise at least one group which is hydrolysable under physiological conditions.

The hydrogels of the present invention can easily be tailored with respect to their degradation kinetics, which enables a tremendously expanded toolbox for the controlled delivery of highly bioactive compounds, such as protein drugs. Especially, in the case of biological response modifiers with a narrow therapeutic window, which are useful in the treatment of various diseases wherein the immune system is involved, this is very important.

An increasing degree of substitution (DS; amount of hydrolysable spaces containing crosslinkable branches per 100 main water-soluble polymer residues; determinable by ¹H-NMR) yields a more crosslinked network. This results in a slower swelling rate and, potentially, in a slower degradation time. The swelling rate, on the other hand, may also be influenced by the porosity or initial water content of the hydrogel. Typically, the initial water content of the hydrogels of the invention ranges from about 50 to about 90 or 95 wt.-%. In moat cases, a relatively low initial water content correlates to a relatively high swelling rate and vice versa.

The hydrogels of the present invention can be prepared in such a way that degradation times from less than 1 day up to about 3 months and longer can be obtained. This can for instance be effected by varying the initial water content in the aqueous polymer solution to be crosslinked and the DS. Geld with a high initial water content, such as water contents higher than 85 wt. % predominantly contain intramolecular crosslinks, while lower initial water contents give more intermolecular crosslinking. Gels with less intermolecular crosslinks dissolve faster at the same DS.

Optionally, two or more chemically different prepolymers may be copolymerized to prepare the hydrogel of the invention. Also, it may be useful to include one or more other polymerisable species, such as low molecular weight monomers having at least one polymerisable group, in order to introduce further functionalities into the polymer network of the hydrogel. For example, the copolymerization of a prepolymer and methacrylic acid will lead to the introduction of side chains which render the hydrogel more hydrophilic. Another option to influence the properties of the hydrogel is the use of radical scavengers, such as cysteine, ascorbate, or tocopherol derivatives, to modulate the extent of polymerization.

Drugs can be loaded into hydrogels either by equilibration in a drug-containing solution (see e.g. Kim et al. Pharm. Res. 9(3) (1992); incorporated herein by reference) or by incorporation of the drug during the preparation of hydrogel (see e.g. Heller et al. in Biomaterials 4 (1983) 262-266; incorporated herein by reference).

Loading by equilibration, however, normally leads to a rather low drug content in the delivery system. This is especially the case, when the drug is a macromolecular compound. Unless the pore size of the hydrogel is rather large, the macromolecules will only adhere to the outer surface, which may lead to a burst release.

Therefore, preferably, the drug is loaded during polymerization or crosslinking.

That is, the polymerization, (or copolymerization) and hydrogel formation may take place in the presence of a bioactive compound. As used herein, a bioactive compound is any chemical or biological substance or mixture of substances which is useful for the diagnosis, prevention or treatment of diseases, symptoms, and other conditions of the body, or for influencing a body function. In this context, the terms “active” and “bioactive” may be used interchangeably.

Preferred active compounds are those which are used in chronical or long-term treatment regimen and/or which have a low oral bioavailability, such as hormones, growth factors, hormone antagonists, antipsychotics, antidepressants, cardiovascular drugs, and the like. In another aspect, a preferred class of active compounds is that of peptides and proteins, in particular proteins, which can be delivered effectively with the gel compositions of the invention, providing drug release over extended time periods, thus eliminating the need for the frequent injection of these compounds.

Among the preferred peptides and proteins are erythropoetins, such as epoetin alpha, epoetin beta, darbepoetin, hemoglobin raffimer, and analogues or derivatives thereof; interferons, such as interferon alpha, interferon alpha-2b, PEG-interferon alpha-2b, interferon alpha-2a, interferon beta, interferon beta-1a and interferon gamma; insulins; antibodies, such as rituximab, infliximab, trastuzumab, adalimumab, omalizumab, tositumomab, efalizumab, and cetuximab; blood factors such as alteplase, tenecteplase, factor VII(a), factor VIII; colony stimulating factors such as filgrastim, pegfilgrastim; growth hormones such as human growth factor or somatropin; interleukins such as interleukin-2 and interleukin-12; growth factors such as beclapermin, trafermin, ancetism, keratinocyte growth factor; LHRH analogues such as leuprolide, goserelin, triptorelin, buserelin, nafarelin; vaccines, etanercept, imiglucerase, drotrecogin alpha.

Other preferred active compounds are polysaccharides and oligo- or polynucleotides, DNA, RNA, iRNA, antibiotics, and living cells. Another class of preferred active compounds comprises drug substances acting on the central nervous system, even if they are small molecules and orally bioavailable, for example risperidone, zuclopenthixol, fluphenazine, perphenazine, flupentizol, haloperidol, fluspirilen, quetiapins, clozapine, amisulprid, sulpirid, ziprasidon, etc.

Alternatively, the active compound may be a native living cell, a modified cell, or a plurality of cells. Encapsulated or immobilized cells can potentially be injected or implanted to replace physiological functions which have are absent in a patient due to a specific disease or condition. For example, diabetes patients could be treated with gel-encapsulated Langerhans cells which can produce and secrete insulin. In this application, both the living cells and the insulin could be considered as the active compound.

If the hydrogels are prepared without an active compound being present, they can subsequently be loaded with such an agent, e.g. by incubating them with an aqueous solution of the active compound (in a similar fashion as described e.g. by Kim et al, in Pharm. Res. 9(3) (1992) 283-290; vide supra). Retrospective loading by incubation, however, normally leads to a rather low drug content in the delivery system. This is especially the case when the drug is a macromolecular compound. Unless the pore size of the hydrogel is rather large, the macromolecular drug will only adhere to the outer surface, which may lead to undesirable drug release behavior such as burst release. Therefore, preferably, an active compound is incorporated into the hydrogels during their polymerisation.

In one of the preferred embodiments, the hydrogels are prepared in the form of microparticles. As used herein, microparticles are defined herein as substantially solid or semisolid particles having a weight- or volume average diameter in the region of about 0.1 to about 1.000 μm, but usually of about 1 to about 500 μm, regardless of their specific composition, geometrical shape, or internal structure. For example, spherical microparticles, which are often referred to as microspheres or nanospheres, are included in the term microparticles, just as capsular structures, such as micro- or nanocapsules. Several other synonyms may exist to describe microparticles as defined above.

Microspheres can e.g. be prepared by dissolving or suspending a prepolymer of the invention in an aqueous solvent. The solution or suspension thus obtained can be emulsified in another liquid phase which is at least partially immiscible with water (e.g. silicone oil) to yield a water-in-oil emulsion. Upon the addition of a suitable initiator system, the polymerisable groups of the side units of the prepolymer undergo radical polymerization, leading to the formation of covalent crosslinks, thus yielding a chemical hydrogel in the form of spherical microparticles.

The microparticles can be adapted for topical, oral, rectal, vaginal, or ophthalmic administration; preferably, however, they are adapted for parenteral administration. As used herein, parenteral administration includes any invasive route of administration, such as subdermal, intradermal, subcutaneous, intramuscular, locoregional, intratumoral, intraperitoneal, interstitial, intralesional, with some less preference in the context of this invention also intravenous, intraarterial etc. the most preferred routes of administration of the gel compositions are subcutaneous, intramuscular, and intratumoral.

Accordingly, the microparticles preferably have am average diameter enabling their intramuscular, subcutaneous, or locoregional injection, such as in tike range of about 1 to about 150 μm as measured by laser diffraction, and more preferably from about 25 to about 120 μm. If the microparticles are to be administered via the intravenous route, their average diameter should be below approx. 1 μm. If a pulmonary administration is intended, the microparticles should have an average diameter below approx. 6-7 μm, and more preferably below about 3-4 μm.

The microparticles may be used as drug carriers in pharmaceutical compositions. Such compositions are also provided by the present invention. Typically, a pharmaceutical composition of the invention will comprises the microparticles in sterile, injectable form. preferably, the composition comprises a dry component containing the microparticles, and—optionally—a sterile, aqueous, liquid carrier for reconstituting the microparticle suspension. Both the dry component and the liquid carrier may comprise further physiologically acceptable excipients which are useful for formulating pharmaceutical compositions. Examples of such excipients include stabilizers, bulking agents, matrix forming agents, lyophilisation aids, antioxidants, chelating agents, preservatives, solvents, cosolvents, surfactants, osmotic agents, acidic or alkaline excipients for adjusting the pH, etc.

Being typically adapted for parenteral administration also means that the pharmaceutical compositions of the invention are formulated and processed to meet the requirements of parenteral dosage forms. Such requirements are, for example, outlined in the major pharmacopoeias. In one aspect, the composition, or its premixes or the kits form which the composition is made prior to administration, must be sterile. In another aspect, the excipients must be selected to be safe and tolerable for parenteral administration. In a further aspect, the compositions are formulated to be relatively isotonic (or isoosmotic), such as in the region of about 150 to 500 mOsmol/kg, and preferably in the region of about 250 to 400 mOsmol/kg. Furthermore, the ph should be approximately in the physiological range in order to avoid pain and local intolerance upon injection. Preferably, the pH of the composition is in the region of about 4 to 8.5, and more preferably in the region of about 5.0 to 7.5.

As indicated herein-above, the active compound can be a protein drug. However, it is also possible to encapsulate active compound-containing nanoparticles, lipid complexes, micelles, mixed micelles, liposomes, iscoms, or other colloidal carrier systems known to the person skilled in the art. The encapsulation of these nanoparticles has the advantage of further decreasing any burst release of the encapsulated compound.

When a hydrogel according to the invention, such as a hydrogel microsphere, is introduced into a physiological environment, e.g., injected intramuscularly, the hydrogel becomes gradually biodegraded in that the hydrolysable groups or bonds present in the crosslinking units are cleaved. This process eventually results in the formation of a polysaccharide, such as dextran, and of a polyacrylic acid derivative, such as polyhydroxyethylmethacrylate; if the crosslinking units contain lactic, glycolic, and/or succinic acid units, these units will also be eventually released as monomeric species. These degradation products are all biocompatible. It is noted that lactic acid and glycolic acid are endogenous compounds. Many polysaccharides including dextran are non-toxic polymers. Also polyhydroxyethylmethacrylate is a well-known biocompatible polymer, which is probably eliminated form the body via the kidneys.

More in detail, the degradation rate of the hydrogel can be adjusted by varying the water content of the hydrogel, the degree of substitution, the type, number and length of hydrolysable groups in the crosslinking units. it has e.g. been found that hydrolysable groups based on glycolic acid units have a higher hydrolytical sensitivity than those based on lactic acid. thus, if glycolate units are present, an accelerated degradation rate of the hydrogel is observed as compared with lactate based crosslinking units.

The effect of the water content of the hydrogel and the degree of substitution is elaborated in the examples following below.

Active compounds having a relatively high molecular weight, such as oligopeptides and proteins, are released form the biodegradable hydrogels of the present invention primarily during the hydrolysis of the hydrogel, although, at least to some extent, release by diffusion through the aqueous pores of the hydrogel may also take place. For active compounds having a relatively low molecular weight, diffusion may represent the predominant mechanism of release.

The hydrolysis behavior of the hydrogel and the time during which active compounds present in the hydrogel system are released can be adjusted to one another so that the release can take place at any level between first order (no degradation of the hydrogel) and zero order release (fully degradation controlled) (see in this respect Example 6, herein-below). This provides evident advantage over hydrogel systems that are not hydrolysable at physiological conditions, but which are hydrolytically rather stable, such as the known dextran-GMA hydrogel system which does not contain any readily hydrolysable groups in its crosslinking units.

If the hydrogel is provided in the form of microparticles, the rate of release also depends on the particle diameter. This size can be adjusted by varying the process parameters involved in the formation of the prepolymer-containing emulsion from which the microparticles are prepared, such as the stirring speed, viscosity of the external phase etc.

An example of a drug-loaded hydrogel system within the present invention is a hydrogel containing the cytokine interleukin-2 (IL-2). IL-2 is a protein drug which can e.g. be used in the treatment of particular types of cancer.

For IL-2 to be therapeutically effective in cancer treatment, prolonged presence of IL-2 at the site of tumor growth is required. This can be achieved either by administering high doses of TL-2 intravenously through frequent bolus injections (see e.g. Rosenberg et al. JAMA 271, (1994) 907-913), by prolonged continuous infusion (see e.g. West et al., N. Engl. J. Med. 316, (1987), 898-905), or by frequently administering low doses of IL-2 intra- or peritumorally (see e.g. Den Otter et al. Anticancer Res. 18, (1993), 2468-2455).

A major disadvantage of the intravenous route is that for obtaining sufficiently high levels of IL-2 at the site of tumor growth, such high doses of IL-2 have to be administered intravenously, that it becomes severely toxic. In contrast, the intra- or peritumoral approach, as developed by Don Otter et al., has proven to be very successful and virtually non-toxic in various transplanted and spontaneous tumors.

A serious problem for application of this form of therapy in human cancer patients, however, is that IL-2 has to be injected intra- or peritumorally 5 to 10 times within 1 to 2 weeks. For many types of cancer this is not-acceptable burden for the patient, like in cases of lung carcinoma, bladder cancer, and gastric cancer. In first attempts to translate the very effective local, low-dose IL-2 treatment to the human cancer clinic, these logistic problems of IL-2 delivery were already run into. The slow-release delivery system of the present invention makes the use of local IL-2 immunotherapy possible.

For the in vivo application hydrogel suspensions (microspheres) will normally contain up to 10⁵ I.U. of IL-2 in 0.5 ml which are released over a period of 5 days (i.e. 2*10⁴ I.U. of IL-2 released per day). The amount of protein released can be determined with sensitive quantitative detection methods (HPLC, ELISA assays). To investigate whether the released IL-2 is still biologically active and to what extent (i.e. what is the effect of these chemical procedures on the specific activity of IL-2), proliferation assays using the IL-2 dependent CTLL cell line can be performed.

The invention will now be explained in more detail, while referring to the following, non-limiting examples.

### EXAMPLES

To obtain a dextran hydrogel, first a polymerisable methacrylate group was introduced in dextran. For all reactions described below, dextran from Fluka (T40, Mn=16.000, Mw=32.500 g/mol) was used. Various different dextran derivatives were synthesized in which the methacrylate ester was coupled via a spacer to dextran. The spacer contains different hydrolyzable bonds (carbonate and/or carboxylic ester). The degradation behavior of gels prepared from these derivatives was compared with dextran gels derived from glycidyl methacrylate (dex-GMA). In this reference compound, the methacrylate ester is directly coupled to a hydroxyl group of dextran. This reference gel degrades extremely slowly.

### Example 1

### Synthesis of Dex-HEMA

Dextran derivatized with hydroxyethyl methacrylate (dex-HEMA) was synthesized by coupling carbonyldiimidazole (CDI) activated HEMA (HEMA-CI) to dextran.

CDI (1.62 g; 10 mmol) was dissolved in about 10 ml anhydrous tetrahydrofuran (THF). This solution was added to a solution of HEMA (1.30 g; 10 mmol) in 5 ml anhydrous THF. The reaction mixture was stirred for 16 hours at room temperature. After evaporation of the solvent a slightly yellow liquid was obtained (yield 2.93 g). The crude product was dissolved in ethyl acetate, extracted with water to remove imidazole and unreacted HEMA and dried on anhydrous MgSO4. After filtration, the solvent was evaporated and almost pure hydroxyethyl methacrylate activated with CDI (HEMA-CI) was obtained. The structure of this product was confirmed by NMR and IR spectroscopy.

Varying amounts of HEMA-CI (0.73, 1.49, or 2.19 g; 96% pure) were added to a solution of dextran (10 g, 62 mmole glucose units) and N,N-dimethylaminopyridine (DMAP; 2 g, 16.4 mmol) in anhydrous dimethylsulfoxide (DMSO; 90 ml). These reaction mixtures were stirred for 4 days at room temperature after which the reaction was terminated by the addition of about 2 ml of concentrated HCl. The reaction mixture was dialyzed against water for 3 days at 4° C. Dex-HEMA was isolated by lyophilization yielding a white fluffy material (yield>90%). The degree of HEMA substitution was determined by NMR spectroscopy, and amounted 4, 9, and 18, respectively.

### Example 2

### Synthesis of Dex-SA-HEMA

Dextran derivatized with the hemi-ester of succinic acid (SA) and HEMA (dex-SA-HEMA) was synthesized as follows.

SA (2.00 g, 20 mmol), HEMA (2.6 g, 20 mmol), triethylamine (TEA; 0.28 mL 2 mmol) and hydroquinone (inhibitor, +/−10 mg) were dissolved in about 30 ml anhydrous THF. The reaction mixture was stirred for 2 days at 45° C., after which the solvent was evaporated. A yellow liquid was obtained (yield 4.88 g). The structure of HEMA-SA was confirmed by NMR and IR spectroscopy.

HEMA-SA (0.99 g (94% pure), 4 mmol) and dicyclohexylcarbodiimide (DCC; 0.83 g, 4 mmol) were dissolved in about 20 ml anhydrous DMSO. After 15 minutes a precipitate was formed (dicyclohexylureum; DCU) and this mixture was added to a solution of dextran (2.57 g, 16 mmol glucose units) and TEA (0.56 mL 4 mmol) in anhydrous DMSO (20 ml). The resulting mixture was stirred for 3 days at room temperature, after which 3 drops of concentrated HCl were added to terminate the reaction. Next, the reaction mixture was filtered to remove DCU and dialyzed for 3 days at 4° C. After lyophilisation, a white fluffy product was obtained (yield 2.78 g). The degree of substitution was established by NMR spectroscopy and amounted to 8.

### Example 3

### Synthesis of Dex-Lactate-HEMA

Dextran derivatized with HEMA-oligolactide was synthesized as follows as illustrated in scheme l. Three steps can be distinguished.

a. coupling of lactate to HEMA yielding HEMA-lactate;

b. activation of HEMA-lactate using CDI yielding HEMA-lactate-CI

c. coupling of HEMA-lactate-CI to dextran.

A mixture of L-lactide (4.32 g; 80 mmol) and HEMA (3.90 g; 30 mmol) was Heated to 110° C. Thereafter, a catalytic amount of stannous octoate (SnOct₂; 121.5 mg, 0.3 mmol) dissolved in about 0.5 ml toluene was added. The resulting mixture was stirred for 1 hour. After cooling down to room temperature, the mixture was dissolved in THF (20 ml). This solution was dropped in water (180 ml) and the formed precipitate was isolated by centrifugation. The pellet was taken up in ethyl acetate (40 ml), centrifuged to remove solid material, dried (MgSO₄) and filtered. The solvent was evaporated yielding a viscous oil (3.74 g, 45%). The product (HEMA-lactate) was characterized by NMR and IR spectroscopy.

HEMA-lactate (3.74 g, 10.8 mmol) was added to a solution of CDI (1.76 g, 10.8 mmol) in THF and stirred for 16 hours at room temperature. The solvent was evaporated under reduced pressure yielding a viscous oil. The product containing HEMA-lactate-CI and imidazole as major compounds (NMR analysis) was used without further purification.

To a solution of dextran (10 g, 62 mmol glucose units) and DMAP (2.0 g, 10.6 mmol) a varying amount of HEMA-lactate-CI was added (1.61, 3.28 or 4.84 g respectively, 80% pure). These mixtures were stirred for 4 days at room temperature after which the reaction was terminated by the addition of about 2 ml of concentrated HCl. The solutions were dialyzed against water for 2 days. After lyophilization, white fluffy products were obtained (yield around 85%). The degree of substitution (as determined by NMR spectroscopy) amounted to 3, 6 and 10 for the three products, respectively.

Using similar procedures, the length of the lactate spacer can be varied by changing the molar ratio of HEMA and lactide in the first reaction.

### Example 4

### Synthesis of Dex-Glycolide-HEMA

Dex-glycolide-HEMA was synthesized according to the same procedure as described in Example 8 replacing lactide by glycolide.

### Reference Example 1

### Dex-GMA

Dex-GMA was synthesized as described by Van Dijk-Wolthuis et al., Macromolecules 28, (1995), 6317-6322. A reinvestigation of the obtained dextran derivative revealed that the methacrylic ester group is directly coupled to one of the hydroxyl group of dextran, meaning that the glyceryl spacer is not present.

### Example 5

### Preparation of Dextran Hydrogels

Hydrogels were obtained by a free radical polymerization of aqueous solutions of methacrylated dextran (prepared according to Examples 1-4 and Ref. Example 1. Methacrylated dextran (100 mg) was dissolved in 760 μl PBS buffer (10 mM phosphate, 0.9% NaCl, 0.02% NaN₃, pH 7.2). To this solution 90 μl of potassium peroxydisulfate (BPS; 50 mg/ml) in the same buffer was added per gram solution and mixed well. Subsequently, N,N,N′N′-tetramethylethylenediamine (TEMED; 50 μl; 20% (v/v) in water, pH adjusted to 7) was added and the resulting solution was quickly transferred into an Eppendorf tube and polymerized for 1 hour at room temperature yielding a hydrogel material with an initial water content of about 90% (w/w) after polymerization.

The gels were removed from the tubes, accurately weighed and incubated in PBS at 37° C. Periodically, the weight of the gels was determined and used to calculate the degree of swelling (=Wₜ/W₀, in which Wₜ is the weight of the gel at time t and W₀ is the initial weight of the gel). The hydrogel degradation (dissolution) time is defined as the time at which the swelling ratio=0 (or Wₜ=0).

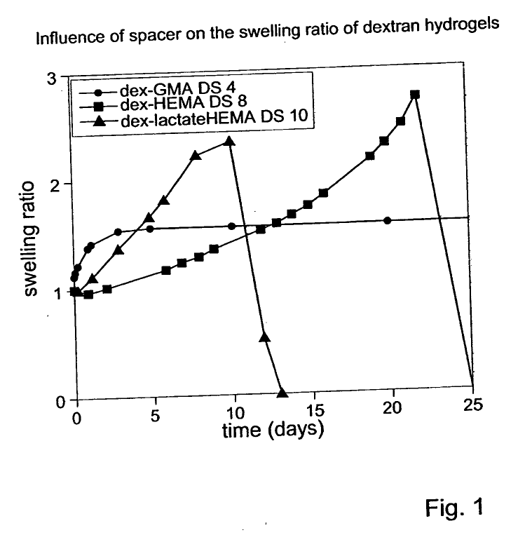

FIG. 1 shows the swelling behavior of three different dextran hydrogels (initial water content 90%). The dex-GMA (DS=4) reached an equilibrium swelling within 3 days; thereafter the weight of the gels remained constant indicating that no significant hydrolysis of methacrylate esters occurred. Dex-HEMA and dex-lactate-HEMA showed a progressive swelling in time until these gels dissolved completely. This demonstrates that in these hydrogel systems hydrolysis of carbonate esters (dex-(lactate)HEMA) and/or lactate esters (dex-lactateHEMA) occurred.

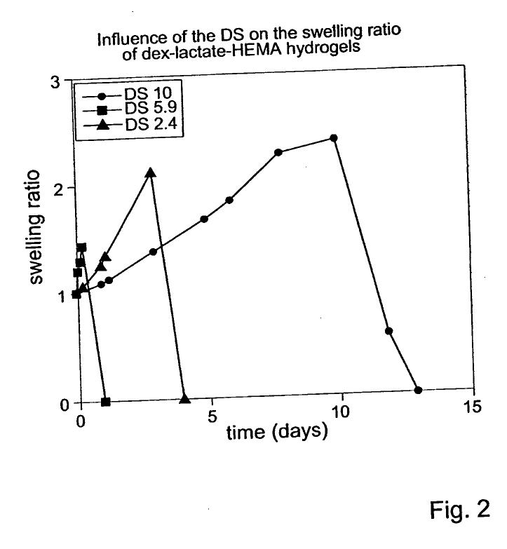

FIG. 2 shows the swelling behavior of dex-lactateHEMA hydrogels (initial water content 92%) and varying degree of substitution. As can be seen an increasing DS resulted in an increasing dissolution time.

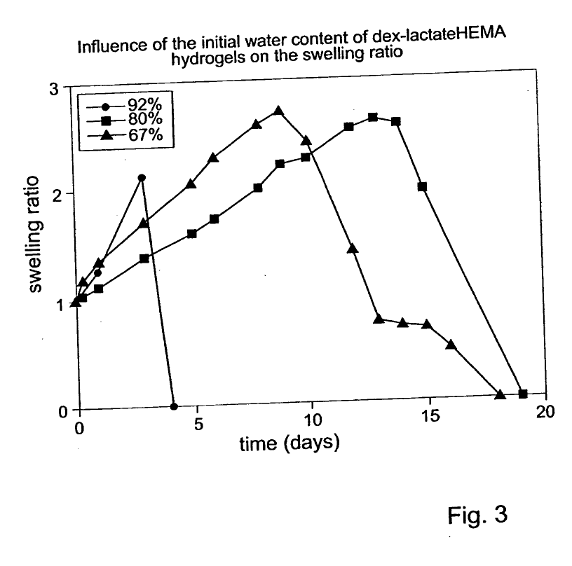

FIG. 3 shows the swelling behavior of dex-lactateHEMA hydrogels with a fixed DS (6) and varying initial water content. It appears that the dissolution time increases with an increasing initial water content.

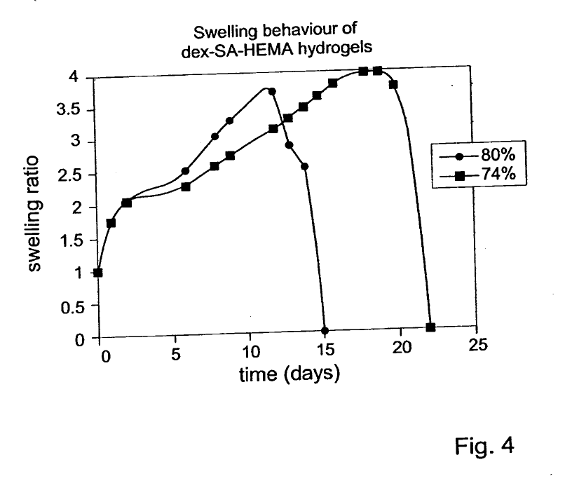

FIG. 4 shows the swelling behavior of dex-SAHEMA hydrogel (DS 3) and a varying initial water content.

The following tables give an overview of the dissolution times of different dextran hydrogels.

Dissolution time (days) of dex-lactate-HEMA hydrogels

| Initial water content | DS 10 | DS 6 | DS 2.5 |
|---|---|---|---|
| 90% | 8-13 | 4 | 1-2 |
| 80% | 16 | 7-18 | 3-9 |
| 70% | 22 | 10-19 | 6-10 |

Dissolution time (days) of dex-HEMA hydrogels

| Initial water content | DS 20 | DS 17 | DS 8 |
|---|---|---|---|
| 92% | 55 | 42 | 25 |
| 80% | ND | 100 | 53 |
| 67% | ND | >100 | 70 |

Dissolution time (days) of dex-HEMA-SA hydrogels

| Initial water content | DS 3 |
|---|---|
| 80% | 15 |
| 74% | 23-28 |
| 60% | 44 |

The dissolution time of the hydro gels (initial water content 92%) obtained by polymerization of dextran derivatized with GMA (DS 4) was about 100 days (pH 7.2, 37° C.). Gels obtained by polymerization of dextran derivatized with GMA (initial water content 80%, DS 11) did not show any signs of degradation (increased swelling) during 70 days, even at extreme conditions (37° C., pH 12 and pH 1).

### Example 6

### Protein Release from Degrading Dextran Hydrogels

The release from non-degrading dex-GMA hydrogels has been studied extensively. It appeared that when the protein diameter was smaller than the hydrogel mesh size, the release of the protein could be effectively described by the free volume theory. In this case the cumulative amount of protein released was proportional to the square root of time (W. E. Hennink, Controlled release of proteins from dextran hydrogels, Journal of Controlled Release 39, (1996), 47-55).

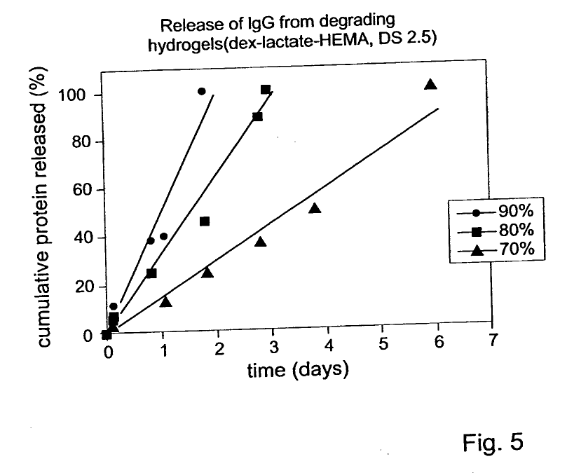

FIG. 5 shows the release of a model protein (IgG) from degrading dextran hydrogels (dex-lactate-HEMA, DS 2.5). The release of IgG from these degrading gels is zero order (cumulative release proportional to time). This is in contrast with the release of proteins from non-degrading dextran hydrogels, where a first order release has been observed.

### Example 7

### Preparation of Activated Hydroxalkylmethacrylamides and Hydroxalkylmethacrylamide-Lactates

Under a nitrogen atmosphere, CDI was dissolved in about 10 ml dichloromethane (DCM) per gram of CDI. After dissolution of CDI, the specified amount of hydroxyethylmethacrylamide (HEMAm), hydroxypropylmethacrylamide (HPMAm), HPMAm-lactate or HPMAm-dilactate was added. HEMAm, HPMAm-lactate and HPMAm-dilactate were added to CDI in a molar ratio of 1:3, whereas HPMAm reacted with CDI in a 1:5 molar ratio. The quantity of the components for each formulation is specified in the following table.

| | | | Compound |
|---|---|---|---|
| Compound | CDI (g) | DCM (mL) | (g) |
| HEMAm | 28.80 | 300 | 7.10 |
| HPMAm | 11.51 | 125 | 2.02 |
| HPMAm-lactate | 6.58 | 125 | 2.30 |
| HPMAm-dilactate | 2.31 | 50 | 1.50 |

The mixture was stirred for one hour at ambient temperature under nitrogen. DCM was then washed with an equal volume of water, and the water layer twice with DCM. The pooled DCM layers were dried with MgSO₄ and subsequently filtered. After addition of a small amount of hydroquinone monomethylether, the solvent was evaporated, yielding the CI-activated compound typically as slightly yellow liquid.

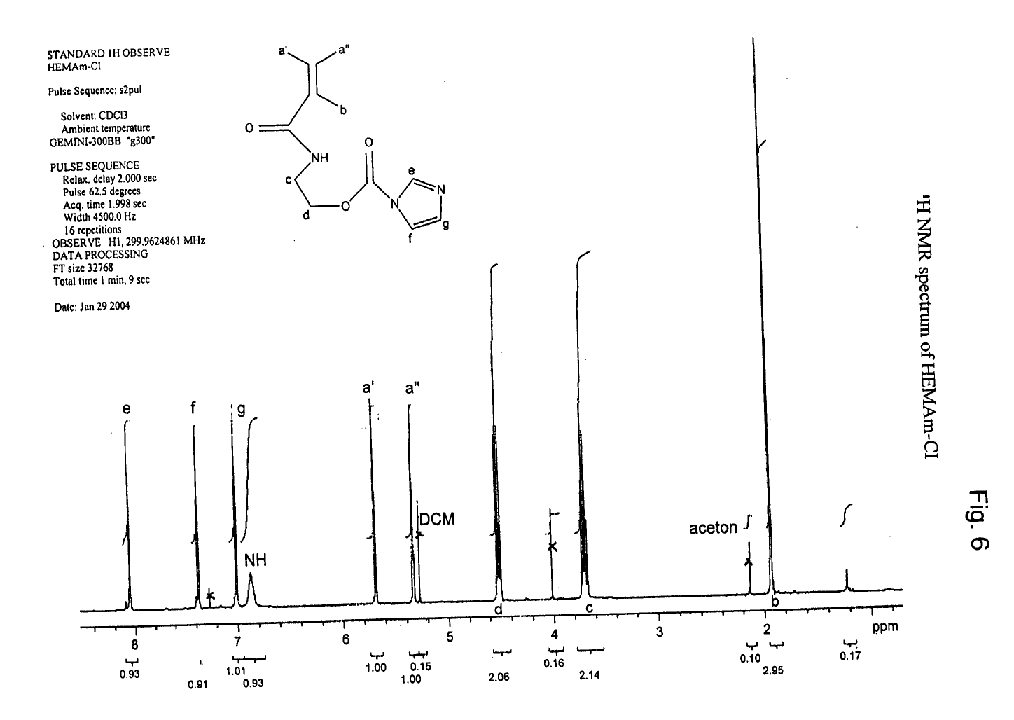

The activation and the purity of the activated compounds were determined by ¹H NMR spectroscopy. As an example the ¹H NMR spectrum of HEMAm-CI (FIG. 6) is given. For the percentage of activation the ¹H NMR spectrum of the activated compound was investigated upon the presence of non-activated compound. Hence, the spectrum of HEMAm-CI was investigated for the presence of free HEMAm. The spectrum of HEMAm-CI showed a downfield shift of the proton at position d compared to the original spectrum and at the original position of 3.75 ppm no peak belonging to this proton was observed anymore. Therefore, the activation percentage of HEMAm-CI was assumed to be 100%. In fact, the percentage of activation was found to be 100% for all compounds. The yields were 6.9 g for HEMAm-CI, 2.47 g for HPMAm-CI, 2.76 g for HPMAm-lactate-CI, and 1.8 g for HPMAm-dilactate-CI.

### Example 8

### Preparation of Prepolymers from Dextran and Activated Hydroxyalkylmethacrylamides and Hydroxyalkylmethacrylamide-Lactates

The CI-activated compounds prepared according to example 7 were used to modify dextran into prepolymers of the invention. For this purpose, dextran was dissolved in DMSO (450 mL per 50 g of dextran) in a dry, stoppered round bottom flask under a nitrogen atmosphere. Subsequently, 4-(N,N-dimethylamino)pyridine (DMAP, 10 g per 50 g of dextran) was dissolved in this solution followed by the addition of HEMAm-CI, HPMAm-CI, HPMAm-lactate-CI or HPMAm-dilactate-CI. The amounts of reagents are specified in the following table.

| | Activated | Dextran | DMSO | | Compound |
|---|---|---|---|---|---|
| Code | compound | (g) | (mL) | DMAP (g) | (g) |
| A | HEMAm-CI | 21.45 | 200 | 4.29 | 2.69 |
| B | HEMAm-CI | 17.44 | 200 | 3.53 | 4.21 |
| C | HPMAm-CI | 9.65 | 100 | 1.94 | 2.47 |
| D | HPMAm- | 6.89 | 100 | 1.39 | 2.76 |
| | lactate-CI | | | | |
| E | HPMAm- | 3.80 | 50 | 0.72 | 1.80 |
| | dilactate-CI | | | | |

The reaction mixture was stirred for 7 days at room temperature under nitrogen flow. Formation of the prepolymer was controlled by determination of the DS by ¹H NMR spectroscopy. For this purpose, the product (0.5 ml) had to be precipitated in 10 ml absolute methanol. The precipitate was isolated by vacuum filtration, rinsed with methanol and DOM and dried in the oven before ¹H NMR spectroscopy in DMSO-d6 was performed.

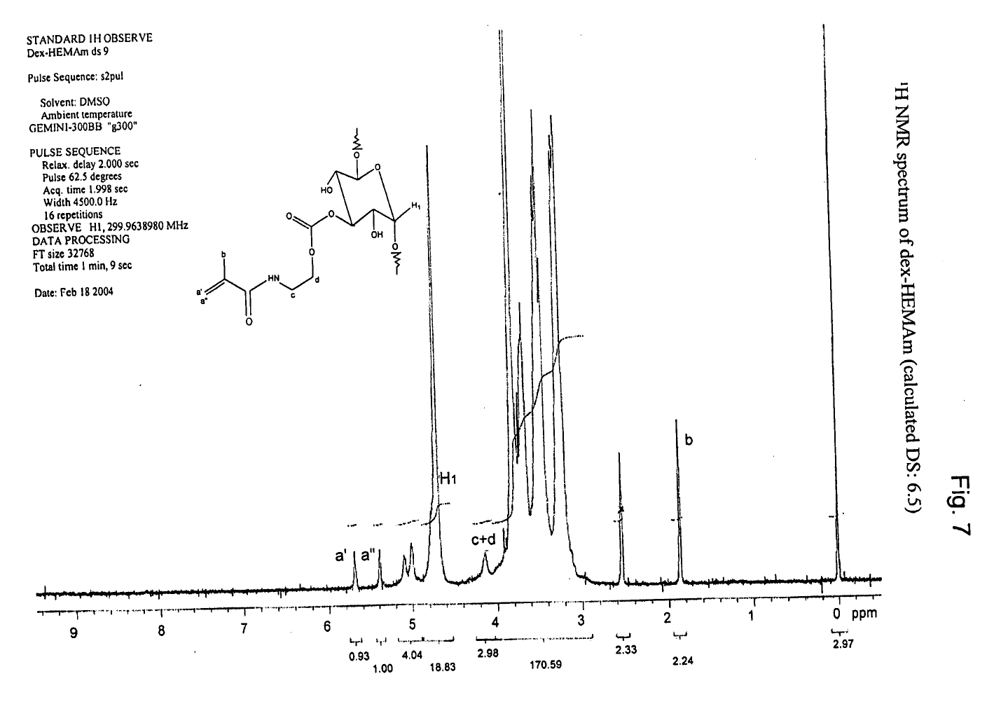

Then, to neutralize DMAP, an equimolar amount of concentrated HCl (37%) was added to the remaining reaction mixture. The solution was transferred to a dialysis tube, and dialyzed for 6 days against reversed osmosis water at 4° C. After dialysis, the solution was filtered using a Buchner filtration system and the obtained product freeze-dried. Soft pieces of white crystals were obtained after freeze-drying. The DS of the resulting dextran derivative was determined by ¹H NMR spectroscopy in DMSO-d6. As an example, the ¹H NMR spectrum of the prepolymer dex-HEMAm with DS 9 required is shown in FIG. 7. The obtained DS values (a=before freeze drying, b=after freeze drying) and the yields are presented in the table below.

| Code | Prepolymer | DS (a) | DS (b) | Yield (g) |
|---|---|---|---|---|
| A | dex-HEMAm | 6.5 | 5.1 | 17.32 |
| B | dex-HEMAm | 19.3 | 11.0 | 17.37 |
| C | dex-HPMAm | 4.3 | 4.4 | 7.79 |
| D | dex-lactide- | 7.0 | 7.4 | 6.37 |
| | HPMAm | | | |
| E | dex-dilactide- | 11.5 | 11.9 | 3.66 |
| | HPMAm | | | |

### Example 9

### Preparation of Degradable Hydrogels from Dextran-Hydroxyalkyl Methacrylamide and Dextran-Lactate-Hydroxyalkyl Methacrylamide Prepolymers

Macroscopic, approx., cylindrically shaped hydrogel unite were prepared from the prepolymers obtained according to example 8 by radical polymerization. In brief, 450 mg of prepolymer (corresponding to a final concentration of 30% (w/w)) was dissolved in 945 μL phosphate buffer (PB, 10 mM, pH 7.2) in a 2 mL Eppendorf cup. To this solution 30 μL of a 20% (v/v) solution of N,N,N′,N′-Tetramethylethylenediamine (TEMED) in 2 M HCl was added. Subsequently, 75 μL of KPS in PB (20 mg/mL) was added, which started the polymerization—or crosslinking—reaction. The solution was again mixed carefully and kept for 1 hour at room temperature. After polymerization, a macroscopic hydrogel unit with a water content of about 70% (w/w) was obtained.

### Example 10

### Degradation of Hydrogels from Crosslinked Dextran-Hydroxyalkyl Methacrylate, Dextran-Hydroxyalkyl Methacrylamide, and Dextran-Lactate-Hydroxyalkyl Methacrylamide Prepolymers

The degradation behavior of the cylindrical hydrogel forms obtained according to examples 5 and 9 and was studied in vitro under physiological conditions, i.e. at 37° C. and pH 7.2 under normal pressure. The hydrogel forms were removed from the Eppendorf cups, placed in pre-weighed 15 mL plastic Greiner tubes and accurately weighed (W₀). The tubes were filled with appropriate 100 mM phosphate-buffered saline (PBS), pH 7.2, containing 0.03 M NaCl and 0.02 N NaN₃, and placed in an oven at 37° C. At regular time intervals, the weight (Wₜ) of the hydrogel forms was determined and used to calculate the swelling ratio defined as Wₜ/W₀. At the same time, the buffer in the tubes was sampled and replaced by fresh PBS.

In all cases, there was an initial increase of weight, indicating the water uptake, or swelling, of the hydrogels, up to a maximal Wₘₐₓ/W₀ (at tₘₐₓ) followed by a weight decrease indicating hydrogel degradation, usually down to the virtual disappearance of the gel (at tₘᵢₙ). The results are shown in the table below and demonstrate how the hydrophilicity and the degradation behavior of the hydrogels of the invention, both of which may influence the in vivo behavior including the drug release behavior, can be adjusted by using different prepolymer side units and DS.

| | Activated | | | | |
|---|---|---|---|---|---|
| Code | compound | DS | tmax (d) | Wmax/Wo | tmin (d) |
| A | dex-HEMAm | 5.1 | 18 | 3.3 | 24 |
| B | dex-HEMAm | 11.0 | 28 | 2.9 | 136 |
| C | dex-HPMAm | 4.4 | 74 | 1.8 | >136 |
| D | dex-lactide- | 7.4 | 19 | 3.3 | 43 |
| | HPMAm | | | | |
| E | dex-dilactide- | 11.9 | 7 | 4.5 | 15 |
| | HPMAm | | | | |
| F | dex-HEMA | 5.0 | 23 | 2.5 | 36 |
| G | dex-HEMA | 7.2 | 20 | 2.3 | 47 |
| H | dex-HEMA | 12.0 | 25 | 2.3 | >136 |

Apparently, for example, dex-HEMAm hydrogels are more hydrophilic than corresponding dex-HEMA hydrogels having a similar DS, and swell faster and to a higher degree. Also, the introduction of one or more lactate units in the crosslinking units leads to a pronounced increase in swelling rate and extent, but also accelerated degradation.

### Example 11

### Release of BSA from Macroscopic Hydrogel Cylinders

Bovine serum albumin (BSA) was used as a model bioactive compound to be incorporated into the hydrogels of the invention. Macroscopic hydrogel cylinders were prepared in analogy to those of examples 5 and 9, but in the presence of BSA during the crosslinking, i.e. polymerization step. More precisely, 750 μL of a 100 mg/mL. BSA solution in PBS was added to the polymer solution prior to polymerization, yielding a loading of 50 mg BSA per gram of hydrogel.

The release of BSA from the hydrogels was determined after incubation of the protein loaded gels at 37° C. in plastic Greiner tubes containing 15 mL of PBS (100 mM, pH 7.2) and 0.03 M NaCl and 0.02 N NaN₃. The tubes were gently shaken, and 3 mL samples were taken periodically and replaced by fresh buffer. Two controls were added to this study. The first control was a dex-HEMA (DS 12) hydrogel without protein, incubated in 15 mL PBS (100mM, pH 7.2) and 0.03 M NaCl, 0.02 N NaN₃ and 75 mg BSA. The second control consisted of 15 mL of PBS (100 mM, pH 7.2) and 0.03 M NaCl, 0.02 N NaN₃ and 75 mg BSA without any hydrogel. The BSA concentration was determined by High-Performance Size Exclusion Chromatography (HPSEC). The encapsulated amount of protein was corrected for the initial weight of the gel.

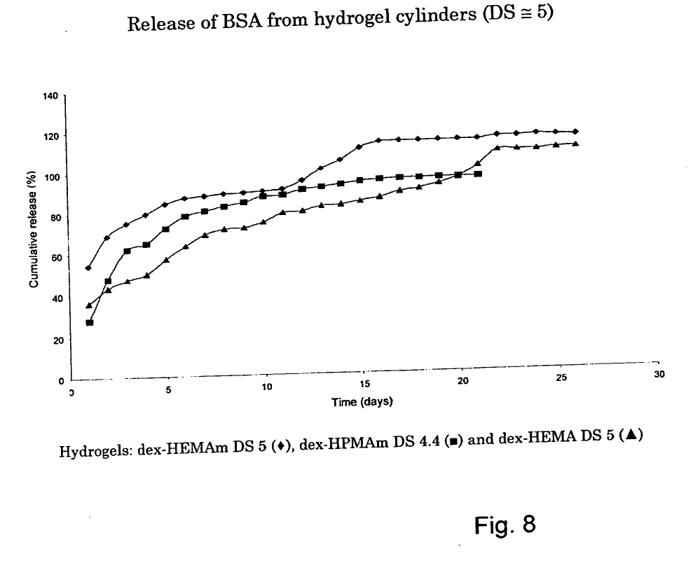

In FIG. 8, the cumulative release of BSA from dex-HEMAm (DS 5), dex-HPMAm (DS 4.4) and dex-HEMA (DS 5) hydrogels is represented. The swelling of these gels was comparable during the first 4 days of the study. It is assumed that, during this phase, the increase in pore size due to swelling and degradation is small, resulting in a small increase in the diffusion of BSA. The release of BSA from dex-HPMAm gels gradually leveled off as this gel degraded only vary slowly. For dex-HEMA gels a more linear release was observed. This increase in BSA release could be due to the gradual swelling and degradation of dex-HEMA gels. As swelling proceeds, it is believed that the pore size of the gel network increases. During the same period, the effective water content increases resulting in an increased diffusion rate. For dex-HEMAm, a biphasic release was observed. A first phase of release of BSA was observed in which the release is perhaps mainly determined by diffusion with minimal increase in pore size, followed by a second phase of accelerated release which could be due to the degradation and collapse of the gel. The maximal release was reached after 16 days for dex-HEMAm and dex-HPMAm gels and after 22 days for dex-HEMA gels, with a release of 113%, 94%, and 108%, respectively. For the first control PBS with 5 mg/mL BSA, the amount of BSA exceeded 100% as well, whereas 100% was reached but not exceeded for the second control, an unloaded gel in PBS with 5 mg/mL BSA. The high values may be ascribed to the evaporation of the incubation buffer as a result of frequent sampling.

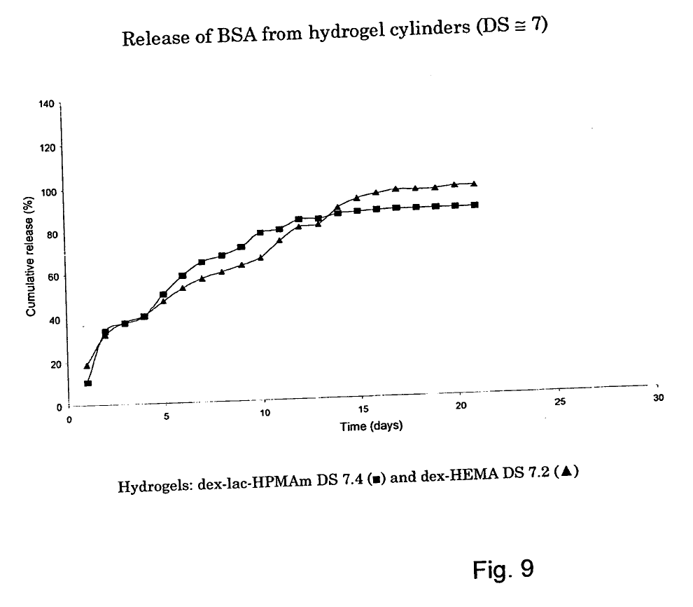

FIG. 9 shows the cumulative release of BSA from dex-lac-HPMAm (DS 7.4) and dex-HEMA (DS 7.2). The maximal release was reached at day 15 for dex-lac-HPMAm and at day 20 for dex-HEMA. The release profile of dex-HEMA appears somewhat closer to a linear release characteristic than that of the dex-lac-HPMAm.

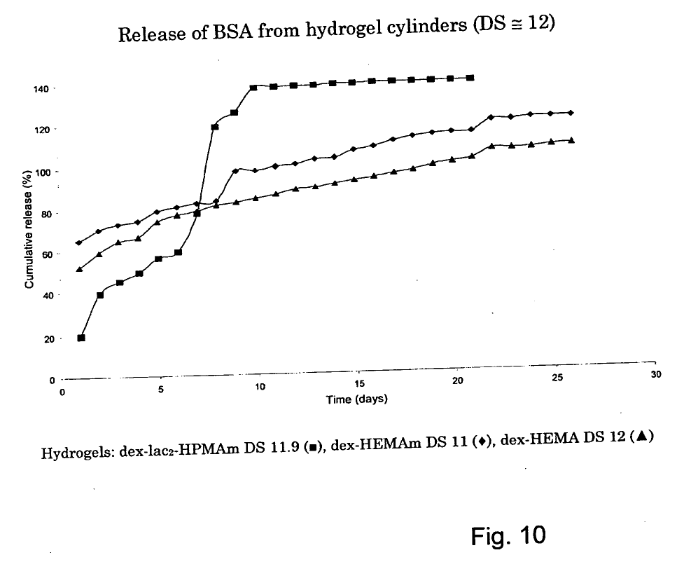

FIG. 10 depicts the cumulative release of BSA from dex-HEMAm (DS 11), dex-lac₂-HPMAm (DS 11.9) and dex-HEMA (DS 12) hydrogels. Interestingly, the dex-lac₂-HPMAm gel initially released BSA more slowly than the dex-HEMAm and dex-HEMA gels. After day 6, however, a rapid increase of BSA release was observed which, most likely, coincides with substantial gel hydrolysis. Maximum release was found at day 10, whereas dex-HEMAm and dex-HEMA gels reached their maximal release at day 22, respectively.

In summary, the release profiles demonstrate that the hydrogels of the invention be tailored to achieve various types of release profiles

1. A prepolymer comprising polymerisable macromolecules, each polymerisable macromolecule comprising a polysaccharide backbone and at least two side units, wherein each of the at least two side units comprises a polymerisable group which is connected to the backbone via at least one group which is hydrolysable under physiological conditions;
wherein the polymerisable group of each of the side units is independently selected from optionally alkylated and/or hydroxy alkylated acrylates and acrylamides; and
wherein the at least one group which is hydrolysable under physiological conditions is selected from carbonate, lactate, glycolate, and succinate.

2. The prepolymer of claim 1, wherein the polysaccharide backbone is a water-soluble polysaccharide or derived from a water-soluble polysaccharide.

3. The prepolymer of claim 2, wherein the water-soluble polysaccharide is a dextran or derived from a dextran.

4. The prepolymer of claim 1, wherein the polymerisable group of each of the side units is independently selected from hydroxyethyl methacrylate, hydroxypropyl methacrylate, hydroxyethyl methacrylamide, and hydroxypropyl methacrylamide.

5. The prepolymer of claim 1, wherein the at least one group which is hydrolysable under physiological conditions is carbonate.

6. The prepolymer of claim 1, wherein at least some of the polymerisable macromolecules comprise at least two side units comprising one or more lactate units.

7. The prepolymer of claim 1, obtained by reacting a polysaccharide with a carbonyl-diimidazole-activated, alkylated and/or hydroxyalkylatad acrylate or acrylamide in the absence of a catalyst.

8. A biodegradable hydrogel comprising a polymeric network, said network comprising polysaccharide backbones which are interconnected through crosslinking units, each of said crosslinking units comprising at least one group which is hydrolysable under physiological conditions;
wherein said polymeric network is obtainable by the polymerization of the prepolymer of claim 1.

9. The biodegradable hydrogel of claim 8, wherein the polysaccharide backbones are composed of, or derived from, a water-soluble polysaccharide.

10. The biodegradable hydrogel of claim 9, wherein the water-soluble polysaccharide is a dextran or is derived from dextran.

11. The biodegradable hydrogel of claim 8, wherein the at least one group which is hydrolysable under physiological conditions is a carbonate group.

12. The biodegradable hydrogel of claim 8, comprising crosslinking units which comprise one or more lactate units.

13. The biodegradable hydrogel of claim 8, farther comprising a bioactive compound.

14. The biodegradable hydrogel of claim 13, wherein the bioactive compound is selected from the group consisting of peptides, proteins, vaccines, oligonucleotides, polynucleotides, hormones, cytostatic or cytotoxic agents, and agents acting on the central nervous system.

15. The biodegradable hydrogel of claim 8, being in the form of microparticles.

16. A pharmaceutical composition comprising the biodegradable hydrogel of claim 8.

17. A biodegradable hydrogel comprising a polymeric network, said network comprising polysaccharide backbones which are interconnected through crosslinking units, each of said crosslinking units comprising at least one group which is hydrolysable under physiological conditions;
wherein said polymeric network is obtainable by the copolymerization of at least two chemically different prepolymers according to claim 1.

18. The biodegradable hydrogel of claim 17, wherein the polysaccharide backbones are composed of dextran or derived from dextran.

19. The biodegradable hydrogel of claim 18, wherein the at least one group which is hydrolysable under physiological conditions is a carbonate group.

20. The biodegradable hydrogel of claim 17, comprising crosslinking units which comprise one or more lactate units.

21. The biodegradable hydrogel of claim 17, further comprising a bioactive compound.

22. The biodegradable hydrogel of claim 21, wherein the bioactive compound is selected from the group consisting of peptides, proteins, vaccines, oligonucleotides, polynucleotides, hormones, cytostatic or cytotoxic agents, and agents acting on the central nervous system.

23. The biodegradable hydrogel of claim 17, being in the form of microparticles.

24. A pharmaceutical composition comprising the biodegradable hydrogel of claim 17.

25. A method for preparing a biodegradable hydrogel, said method comprising the polymerization of a prepolymer according to claim 1.

26. The method of claim 25, wherein said polymerization is conducted in the presence of a low molecular weight compound having at least one polymerisable group.

27. The method of claim 25, wherein said polymerization is conducted in the presence of a bioactive compound.

28. The method of claim 27, wherein the bioactive compound is selected from the group consisting of peptides, proteins, vaccines, oligonucleotides, polynucleotides, hormones, cytostatic or cytotoxic agents, and agents acting on the central nervous system.

29. A method for preparing a biodegradable hydrogel, said method comprising the copolymerization of at least two chemically different prepolymers according to claim 1.

30. The method of claim 29, wherein said copolymerization is conducted in the presence of a bioactive compound.

31. The method of claim 30, wherein the bioactive compound ia selected from the group consisting of peptides, proteins, vaccines, oligonucleotides, polynucleotides, hormones, cytostatic or cytotoxic agents, and agents acting on the central nervous system.
